(19) United States
(12) Patent Application Publication
(10) Pub. No.: US 2021/0380327 A1
(43) Pub. Date: Dec. 9, 2021

(54) HEARING PROTECTION DEVICE CARRY CASE, DISPENSER SYSTEM, APPLICATOR SYSTEM, AND METHOD OF USE

(71) Applicant: Fernando Newcomb, Hong Kong (HK)

(72) Inventor: Fernando Newcomb, Hong Kong (HK)

(21) Appl. No.: 17/335,368

(22) Filed: Jun. 1, 2021

Related U.S. Application Data
(60) Provisional application No. 63/033,869, filed on Jun. 3, 2020.

(51) Int. Cl. B65D 83/00; B65D 83/04

(52) U.S. Cl. CPC B65D 83/005; B65D 83/0409; A61F 11/08; B65D 2583/0481; B65D 83/0038

(57) ABSTRACT

A hearing protection device carry case, dispenser system, applicator system, and methods of use are presented. The present disclosure is directed toward a portable carry case for storage and preparation of sounds blocking components. The present carry case system also includes a dispensing system for moving the sound blocking component into position and then moving the component into an applicator so that the applicator can subsequently and safely place the component into place for a user. The present disclosure provides the state of the art with a carry case having a dispensing system, a unique applicator, and methods of using the system and applicator.

### CROSS-REFERENCE TO RELATED APPLICATIONS:

The present application claims priority to the U.S. Provisional Patent Application No. 63/033,869 which was filed on Jun. 3, 2020, which is hereby incorporated by reference herein in its entirety, including any figures, tables, or drawings.

### FIELD OF THE DISCLOSURE

The present disclosure relates generally to an earplug carry case and dispenser system and method of use. More specifically, the present disclosure is a device with multi-function capability as both a carry case and an all inclusive dispenser and insertion system, and method of use which provides for earplug utilization. The present disclosure is specifically designed to make earplug insertion easy and correct. However, the present disclosure is not limited to these novel and inventive improvements, and it may further be adapted for a variety of purposes.

### COPYRIGHT NOTICE

At least a portion of the disclosure of this patent document contains material that is subject to copyright protection. The copyright owner has no objection to the facsimile reproduction by anyone of the patent document or the patent disclosure, as it appears in the Patent and Trademark Office patent files and/or records, but otherwise reserves all copyright rights whatsoever. The following notice applies to the software and data as described below and in the drawings that form a part of this document: Copyright Sentinel Systems Limited. All rights reserved.

### BACKGROUND OF THE DISCLOSURE

Hearing protection devices such as devices worn over the ear or worn in the ears are well known in the art. A hearing protection device is used to help prevent noise induced hearing loss from hazardous noises created on job sites, at sporting events, at airports, and other places where noise is produced and/or noise is polluted. Hearing protection devices generally reduce (rather than eliminate) the level of noise entering the ear canal.

Many different types of hearing protection devices exist, including earmuffs, earplugs, electronic hearing protection devices, and semi-insert devices. Earmuff style hearing protection devices fit over the outer ear of a user and typically consist of two ear cups and a hand band which secures the ear cups in place. The ear cups are usually lined with a sound absorbing material. Earplugs are designed to fit in the ear canal. Earplugs come in a variety of forms and functions including premolded and formable earplugs. Formable earplugs are pliable and take the form of a user's ear canal. In this way, the formable earplugs block soundwaves from entering the ear canal thus blocking noise and preventing noise-induced hearing loss. The disclosure herein is designed for formable earplugs. However, the disclosure herein may be adapted to other types and designs of hearing devices and should not be limited thereto.

Earplugs are required in many countries to control risk of exposure to loud noise in the workplace. In the United States, the Occupational Safety and Health Administration (OSHA) requires the provision of hearing protection devices. Additionally, other organizations and hearing professionals recommend using hearing protection devices when a person is exposed to noise levels that exceed 85 decibels. Furthermore, if a person is exposed to loud noises or moderate noises over a period of time, hearing protection devices are recommended. Exposure to noise, especially loud noise, or noise over a duration of time, has proven to cause deafness or significant hearing loss. Thus, hearing protection devices are critical in protecting one from hearing loss caused by noise or noise over a duration.

The amount of protection from noise can vary based on the physical fit of a hearing protection device. Additionally, the amount of protection will greatly depend on the correct use of a hearing protection device. Hearing protection devices with accurate placement (an airtight seal) and/or accurate insertion (depth into the ear canal) will provide the best hearing protection from hearing loss caused by noise.

Even if hearing protection devices are readily available to users, there are many challenges to achieving the needed protection. Some of the barriers to various hearing protection devices include adequate use (is the device being used at the appropriate time, in the appropriate way, with the appropriate implementation), comfort of the hearing protection device, convenience of the hearing protection device beyond availability, training in using the hearing protection device, as well as beliefs and knowledge and attitude towards the implemented hearing protection device and other hearing protection devices.

In addition to overcoming these challenges and hurdles for proper implementation of hearing protection devices to overcome hurdles of protecting a user's hearing, individuals, companies and the like must also consider hygiene and care of hearing protection devices. In order to prevent irritation or infection of the ear, reusable hearing protection devices have to be cleaned on a regular basis. For this reason, and oftentimes to avoid liability associated with health conditions caused by unclean hearing protection devices, disposable or single use hearing protection devices are used. Disposable or single-use hearing protection devices are intended for a single use and are not washed for reuse. Additionally, for single use applications, correct implementation becomes even more of a challenge.

Hygiene is just one of many issues that exist in the implementation and use of hearing protection devices. Hearing protection devices are often misused, hard to apply, hard to carry, hard to store, and the like. It is estimated that when available, most of the time an earplug is misused or is stored in a way that damages the earplug itself or compromises the hygiene of the earplug.

It is apparent that a need exists for a novel solution to provide a more hygenic, easy to use, easy to implement, and create a more readily available, more cost effective solution to hearing protection. The present disclosure is directed toward providing such a solution.

### SUMMARY OF THE DISCLOSURE

The purpose of this disclosure is to provide the state of the art with an earplug carry case and dispenser system and method of use. More specifically, the present disclosure is a device with multi-function capability as both a carry case and an all inclusive dispenser and insertion system, and method of use which provides for earplug utilization. The present disclosure is specifically designed to make the correct earplug insertion for proper hearing protection and maximizing the effectiveness of an earplug.

The present disclosure offers convenient and safe storage for a hearing protection device. In this way, a user can have convenient access to hearing protection devices wherever that user may be. The storage solution this disclosure presents combines an effective and convenient storage means that is also easy to access. In one example, the carry case presented herein can be small enough to be carried in a users pocket.

In combination with convenience, the present disclosure offers a hygienic solution of a storage case which prevents infection and other hygienic problems caused by the mishandling and/or the lack of convenience associated with most carry or holding cases. Single use and/or disposable hearing protection devices are very small in size and are easily lost or dropped. This further causes hygienic issues if a hearing protection device is dropped on the ground or other surface which could be contaminated. For this reason, and in addition to the above, the disclosure herein presents a way in which the hearing protection device needs not be touched by human hands when transferring from the case to insertion and/or application of the hearing protection device.

The present disclosure provides a carry case, and an application which improves upon the state of the art. One way in which the carry case improves upon the state of the art is that it provides a means for taking the hearing protection device from the interior of the carry case all the way to insertion in the ear canal without a user having to touch the hearing protection device. In this way, the applicator can be used to pick up the hearing protection device as well as used to insert the hearing protection device into the ear canal of a user. Furthermore, the device herein provides an effective use which ensures the hearing protection device is inserted into the ear canal properly. Improper deployment and/or insertion of the hearing protection device is one of the most common flaws with ineffectiveness of hearing protection devices.

Through a solution that incorporates convenience, hygienic advantages along with safety advantages, as well as proper deployment, the disclosure herein presents a much improved, novel, and needed hearing protection device carry case and method of use.

Said another way, the disclosure consists of three unique elements intended to be claimed separately as well as a method of use and/or application. First, the present disclosure presents a convenient carry case that improves upon the convenience and dispensary advantages of the state of the art. Second, the present disclosure presents a convenient carry case and applicator that improves upon the hygienic state of the art. Third, the present disclosure presents a convenient carry case that improves upon the effectiveness and/or greatly increases the proper and safe use of the disclosed hearing protection device.

Taken individually, all the above provide for effective improvements upon the state of the art. Additionally, taken in combination, this disclosure provides for a convenient, easy to use, hygienic, effective carry case, applicator and method of use that greatly improves upon the state of the art and/or provides a novel device and methodology of using a hearing protection device.

Thus, it is a primary object of the disclosure to provide an earplug carry case and dispenser system, and methods of use that improve upon the state of the art.

Another object of the disclosure is to provide an earplug carry case and dispenser system, and methods of use that are easy to use.

Yet another object of the disclosure is to provide an earplug carry case and dispenser system, and methods of use that are safe.

Another object of the disclosure is to provide an earplug carry case and dispenser system, and methods of use that are hygienic.

Yet another object of the disclosure is to provide an earplug carry case and dispenser system, and methods of use that correctly inserts a hearing protection device within the ear canal of a user.

Another object of the disclosure is to provide an earplug carry case and dispenser system, and methods of use that effectively inserts a hearing protection device within the ear canal of a user.

Yet another object of the disclosure is to provide an earplug carry case and dispenser system, and methods of use that are convenient.

Another object of the disclosure is to provide an earplug carry case and dispenser system, and methods of use that are easy to access.

Yet another object of the disclosure is to provide an earplug carry case and dispenser system, and methods of use that provide quick insertion of a hearing protection device.

Another object of the disclosure is to provide an earplug carry case and dispenser system, and methods of use that provide proper insertion of a hearing protection device.

These and other objects, features, or advantages of the present disclosure will become apparent from the specification and claims.

### BRIEF DESCRIPTION OF THE DRAWINGS:

FIG. 1 is a top, perspective view of the hearing protector device carry case, dispenser system, applicator system and method of use; the view showing the rear side of the system.

FIG. 2 is a top, perspective view of the hearing protector device carry case, dispenser system, applicator system and method of use; the view showing the rear side of the carry case in a closed position.

FIG. 3 is a top view of the hearing protector device carry case, dispenser system, applicator system and method of use; the view showing the carry case in a closed position with the ring or clip extended at least partially.

FIG. 4 is a rear, elevation view of the hearing protector device carry case, dispenser system, applicator system and method of use; the view showing the carry case in a closed position with the ring or clip extended at least partially; the view showing the back of the carry case and the opposing sides of the back.

FIG. 5 is a front, elevation view of the hearing protector device carry case, dispenser system, applicator system and method of use; the view showing the cover, the ridges, the grip, the front of the carry case, and opposing sides of the front.

FIG. 6 is a side elevation view of the hearing protector device carry case, dispenser system, applicator system and method of use; the view showing the side nearest the dispenser system.

FIG. 7 is a side elevation view of the hearing protector device carry case, dispenser system, applicator system and method of use; the view showing the storage chamber side; the storage chamber housing plugs and applicator.

FIG. 8 is a bottom, perspective view of the hearing protector device carry case, dispenser system, applicator system and method of use; the view showing the dispenser system removed from the hollow interior of the carry case; the view showing the main body, the exterior surface, the interior surface, the hollow interior, and a push release feature of the chamber.

FIG. 9 is a top, perspective view of the dispenser system removed from the hollow interior of the carry case; the view showing chambers used for dispensing sound blocking devices; the view showing the dispenser extending from a top to a bottom; the view showing the hollow tubes of the chambers; the view showing a step; the view showing springs and other features.

FIG. 10 is a top view of the dispenser of view of the dispenser system removed from the hollow interior of the carry case.

FIG. 11 is a front view of the dispenser system removed from the hollow interior of the carry case; the view showing a compression clip, spring, and spring support.

FIG. 12 is a rear, elevation view of the dispenser system removed from the hollow interior of the carry case.

FIG. 13 is a side elevation view of the dispenser system removed from the hollow interior of the carry case.

FIG. 14 is a side elevation view of the dispenser system removed from the hollow interior of the carry case.

FIG. 15 is a bottom, perspective view of the dispenser system removed from the hollow interior of the carry case.

FIG. 16 is a top, perspective view of a hearing protection device; the view showing an earplug.

FIG. 17 is a top, perspective view of the hearing protector device carry case, dispenser system, applicator system and method of use; the view showing an exploded view of some of the components of the system; the view showing a main body; the view showing a chamber; the view showing a top lid.

FIG. 18 is a top, perspective view of the hearing protector device carry case, dispenser system, applicator system and method of use; the view showing an exploded view of some of the components of the system; the view showing a main body; the view showing a chamber; the view showing a top lid; the view showing an applicator; the view showing a plurality of hearing protection devices; the view showing how the applicator and protection devices can be stored; the view showing a clip attachment feature for the rear of the main body; the view showing various features of the chamber and ability of chamber to open and close.

FIG. 19 is a top, perspective view of the hearing protector device carry case, dispenser system, applicator system and method of use; the view showing a main body; the view showing the chamber in a closed position; the view showing the dispenser removed from the hollow interior of the main body.

FIG. 20 is a top, perspective view of the hearing protector device carry case, dispenser system, applicator system and method of use; the view showing an exploded view of some of the components of the system; the view showing a main body; the view showing a chamber; the view showing a top lid.

FIG. 21 is a top, perspective view of the hearing protector device carry case, dispenser system, applicator system and method of use; the view showing the chamber in the open position; the view showing the applicator stored within the chamber; the view showing a plurality of earplugs stored within the chamber.

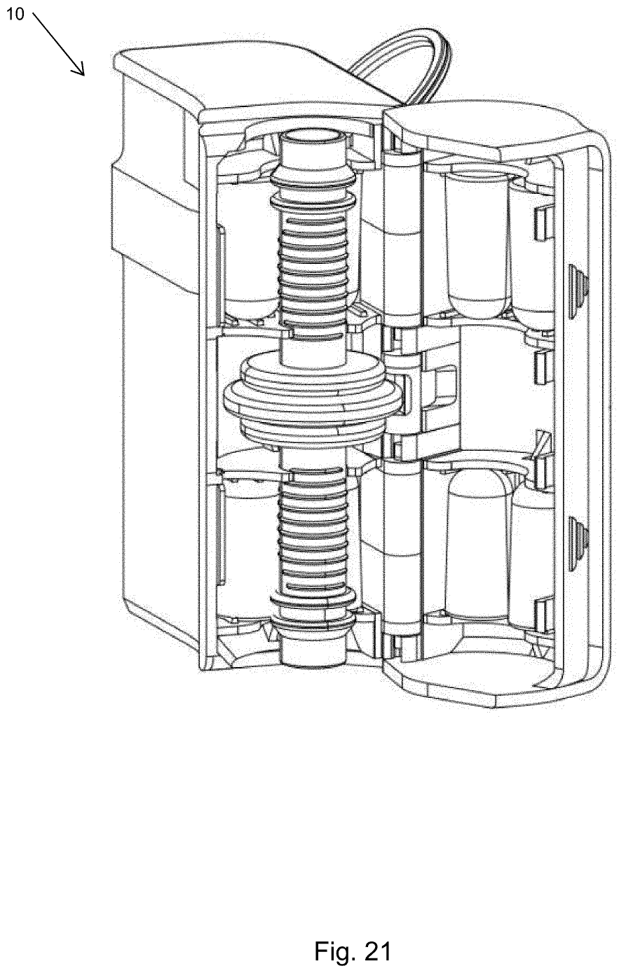

FIG. 22 is a top, perspective view of the hearing protector device carry case, dispenser system, applicator system and method of use; the view showing the front cover; the front cover shown in the closed position.

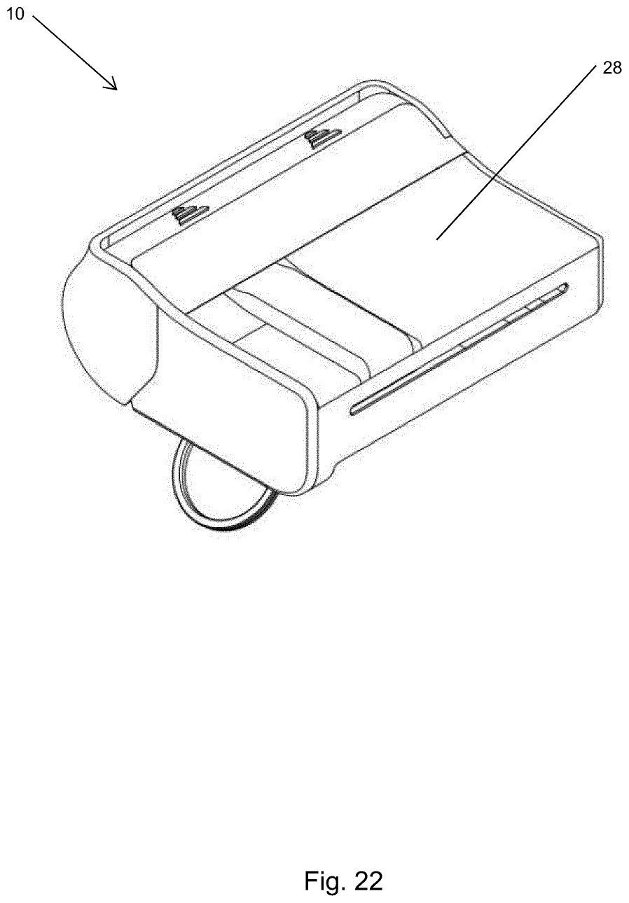

FIG. 23 is a top, perspective view of the hearing protector device carry case, dispenser system, applicator system and method of use; the view showing the front cover; the front cover in an open or at least partially open position; the view showing a plurality of compression clips; the compression clips in the closed position.

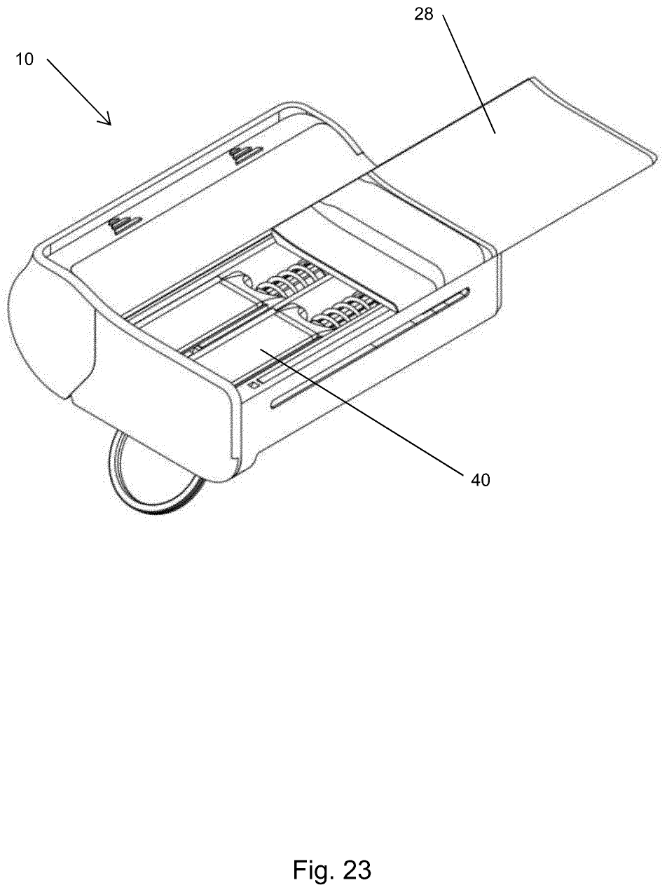

FIG. 24 is a top, perspective view of the hearing protector device carry case, dispenser system, applicator system and method of use; the view showing the front cover; the front cover in the open position; the view showing a plurality of compression clips; the compression clips in the open position.

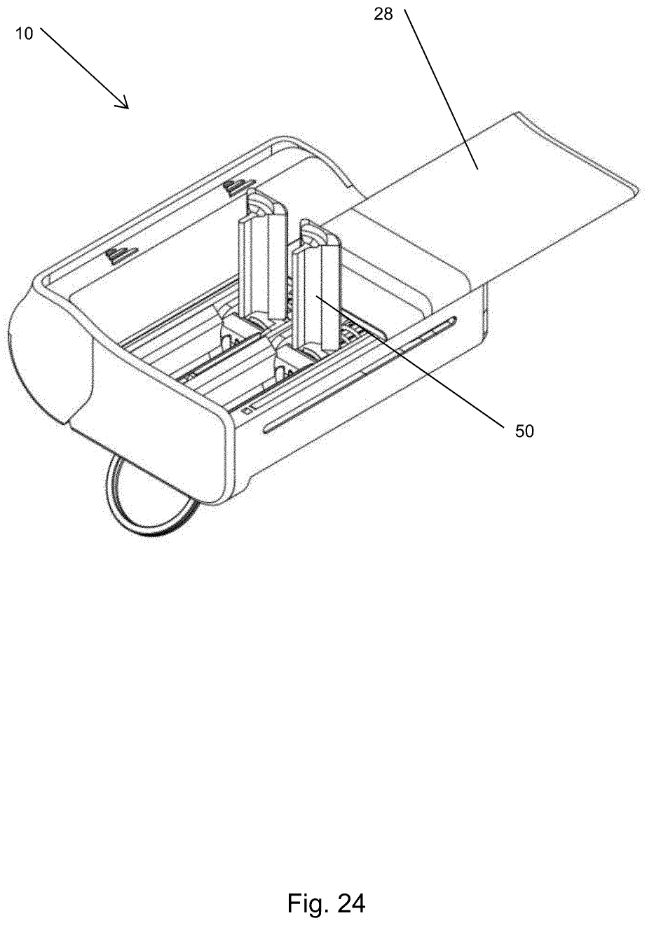

FIG. 25 is a top, perspective view of the hearing protector device carry case, dispenser system, applicator system and method of use; the view showing the front cover; the front cover in the open position; the view showing a plurality of compression clips; the compression clips in the open position; the view showing a plurality of plugs; the view showing the plurality of plugs outside of the chamber.

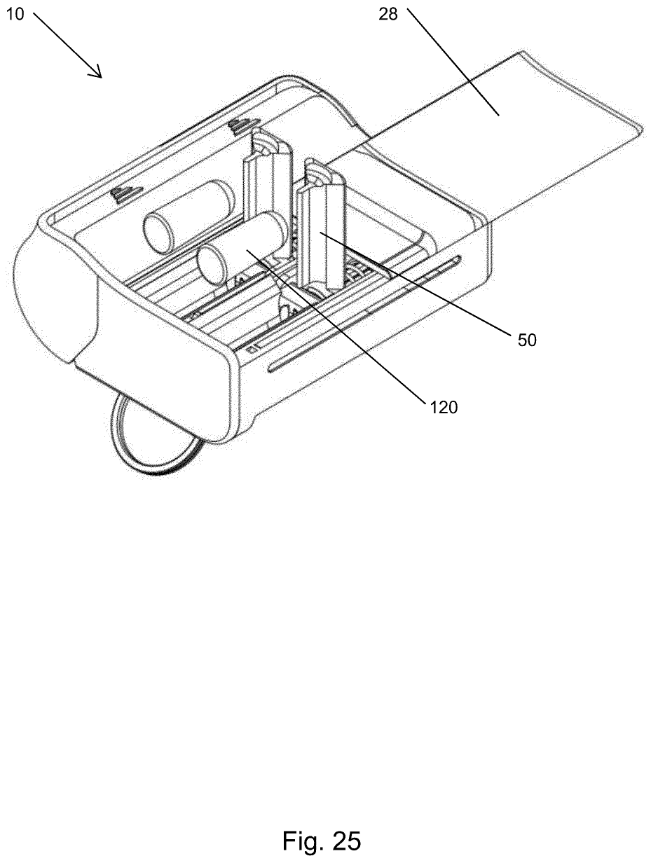

FIG. 26 is a top, perspective view of the hearing protector device carry case, dispenser system, applicator system and method of use; the view showing the front cover; the front cover in the open position; the view showing a plurality of compression clips; the compression clips in a partially open position; the view showing a plurality of plugs; the view showing the plurality of plugs loaded into the chamber.

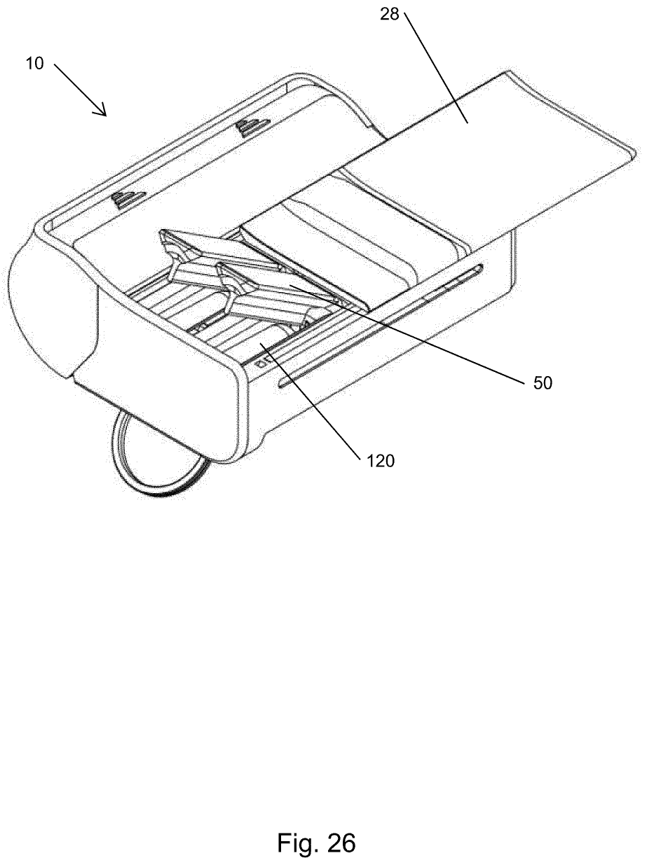

FIG. 27 is a top, perspective view of the hearing protector device carry case, dispenser system, applicator system and method of use; the view showing the lid of the carry case in open position; the view showing the applicator engaged with the carry case; the view showing the applicator in closed position.

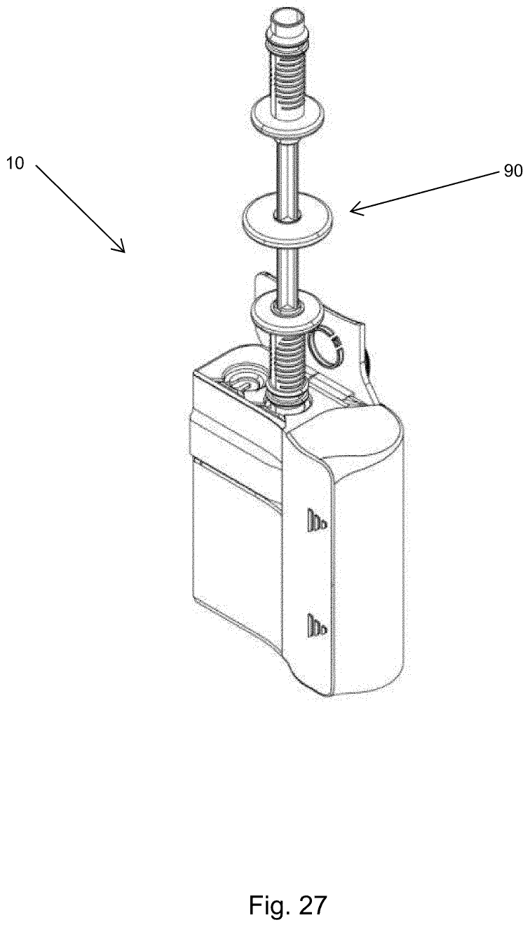

FIG. 28 is a top, perspective view of the hearing protector device carry case, dispenser system, applicator system and method of use; the view showing the lid of the carry case in open position; the view showing the interior of the main body; the view showing the applicator engaged with the carry case; the view showing the applicator in open position.

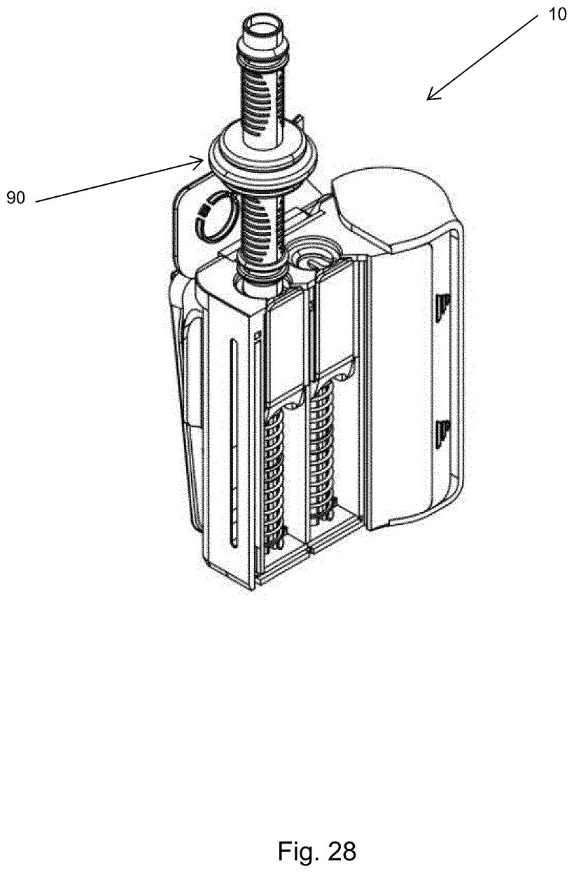

FIG. 29 is a top, perspective view of the hearing protector device carry case, dispenser system, applicator system and method of use; the view showing the lid of the carry case in open position; the view showing the interior of the main body; the view showing the applicator engaged with the carry case; the view showing the applicator in partially engaged position; the view showing the applicator pressing down on the chamber and the spring.

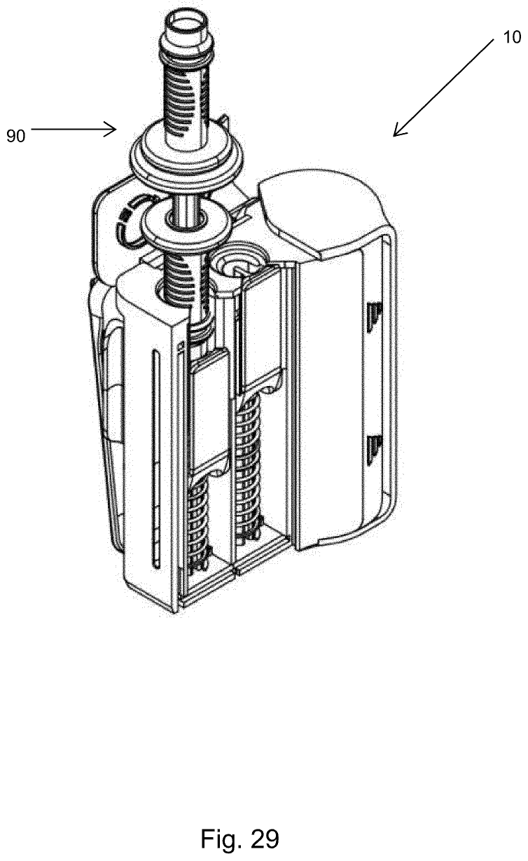

FIG. 30 is a top, perspective view of the hearing protector device carry case, dispenser system, applicator system and method of use; the view showing the lid of the carry case in open position; the view showing the interior of the main body; the view showing the applicator engaged with the carry case; the view showing the applicator in an engaged position; the view showing the applicator pressing down on the chamber and the spring; the view showing the applicator intaking a plug into the hollow interior of the applicator.

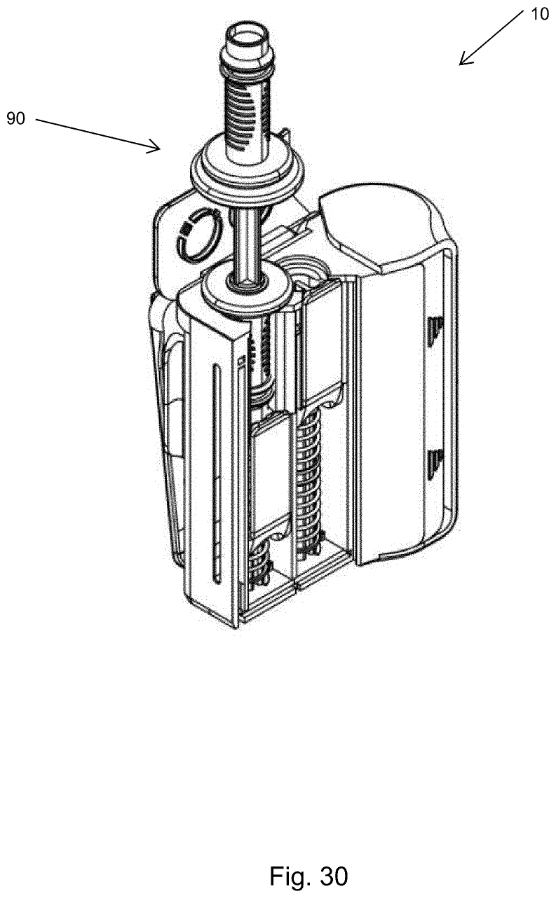

FIG. 31 is a top, perspective view of the hearing protector device carry case, dispenser system, applicator system and method of use; the view showing the the lid of the carry case in open position; the view showing the interior of the main body; the view showing the applicator engaged with the carry case; the view showing the applicator in a loaded position; the view showing the applicator loaded with a plug.

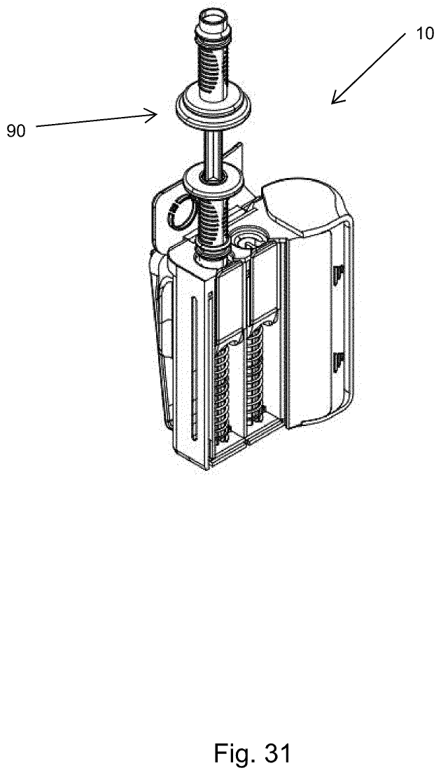

### DETAILED DESCRIPTION OF THE DISCLOSURE:

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural, and other changes may be made without departing from the spirit and scope of the disclosure(s). The following detailed description is, therefore, not to be taken in a limiting sense, and the scope of the disclosure(s) is defined only by the appended claims, along with the full scope of equivalents to which such claims are entitled.

As used herein, the terminology such as vertical, horizontal, top, bottom, front, back, end, sides and the like are referenced according to the views, pieces and figures presented. It should be understood, however, that the terms are used only for purposes of description, and are not intended to be used as limitations. Accordingly, orientation of an object or a combination of objects may change without departing from the scope of the disclosure.

Reference throughout this specification to “one embodiment,” “an embodiment,” “one example,” or “an example” means that a particular feature, structure, or characteristic described in connection with the embodiment or example is included in at least one embodiment of the present disclosure. Thus, the appearance of the phrases “in one embodiment,” “in an embodiment,” “one example,” or “an example” in various places throughout this specification are not necessarily all referring to the same embodiment or example. Furthermore, the particular features, structures, databases, or characteristics may be combined in any suitable combinations and/or sub-combinations in one or more embodiments or examples. In addition, it should be appreciated that the figures provided herewith are for explanation purposes to persons ordinarily skilled in the art and that the drawings are not necessarily drawn to scale.

All illustrations of the drawings are for the purpose of describing selected versions of the present disclosure and are not intended to limit the scope of the present disclosure.

Although the disclosure has been explained in relation to its preferred embodiment, it is to be understood that many other possible modifications and variations can be made without departing from the spirit and scope of the disclosure.

### System

With reference to the figures, a hearing protector device carry case, dispenser system, applicator system, or simply system 10, and method of use are presented herein. System 10 is formed of any suitable size, shape and design and is configured to provide convenient access to a hearing protector device, provide dispensing and safe and easy application of the hearing protector device. In the arrangement shown, as one example, system 10, may comprise a carry case 12, a dispenser 14, an applicator 16, a hearing protector device 18, and methods of use and or methods of application.

In the arrangement shown, as one example, system 10 includes a carry case 12. Carry case 12 is configured to hold a plurality of hearing protector devices 18. Specifically, carry case 12 is configured to hold a plurality of single-use, disposable plugs 120 (or “earplugs”). In this way, the carry case 12 makes access to and transportation of a plurality of hearing protector devices 18 convenient and more hygienic for a user. In this way, use of a hearing protection device to prevent noise-induced hearing loss is increased due to convenience. Additionally, infection and contamination is decreased due to additional hygienic steps and ease of access created by the use of the carry case 12.

In the arrangement shown, as one example, system 10 includes a dispenser 14. In the arrangement shown, as one example, dispenser 14 is configured for rapid and easy dispensing. In the arrangement shown, as one example, this rapid and easy dispensing is done directly into an applicator 16 (to be further discussed herein). Furthermore, in the arrangement shown, as one example, carry case 12 and dispenser 14 are a form of a single construction such that the dispenser can hold a plurality of hearing protector devices 18 in a way in which the hearing protector devices 18 are ready to be dispensed. Additionally, the carry case 12, while holding a plurality of hearing protector devices 18 ready to be dispensed, is also capable of carrying a supply to refill the dispensing device when necessary. In this way, refilling and using is simplified and more readily available to a user.

In the arrangement shown, as one example, the carry case 12 and the dispenser 14 are made of a single piece which is slidably connected such that the carry case 12 opens to provide access to a plurality of plugs 120 stored in the hollow interior 66 of the carry case 12 (to be further discussed herein). While the carry case 12 and the dispenser 14 will be discussed herein as a single component on occasion and shown as a single component, slidably connected, in the figures, it is hereby contemplated that these components may not be a single component or may be connected by another means and in another way.

In the arrangement shown, as one example, system 10 includes an applicator 16. Applicator 16 is configured to accept a hearing protector device 18 therein and subsequently apply the hearing protector device 18 within the ear canal of a user in a way in which the hearing protector device reduces the noise of a surrounding environment. Furthermore, in the arrangement shown, as one example, the carry case 12, dispenser 14, and applicator 16, are all designed to fit conveniently within system 10. In this way, the applicator 16 is readily available for use so that the correct use of a hearing protector device 18 is possible.

### Main Body

In the arrangement shown, as one example, system 10 includes a main body 20. Main body 20 is formed of any suitable size, shape, and design and is configured to house and hold the components of system 10. Main body 20 creates a majority portion of the carry case 12. In the arrangement shown, as one example, main body 20 includes an exterior surface 22, an interior surface 24, a hollow interior 26, a cover 28 (or “front”), a plurality of ridges 29 along the cover, a back 30, opposing sides 32, ridges 33 of the opposing sides 32, a grip 34, the grip having a front 35, the grip having opposing sides 36, and a push release 38, among other components and features.

In the arrangement shown, as one example, the main body 20 is formed of a polymer. However, any other material for forming a useful carry case and dispenser are hereby contemplated for use. Other materials may include enhanced polymers, vinyl, polyvinyl chloride (pvc), metals such as aluminum, steel and the like, metal composites, carbon fiber, as well as other combinations of materials that are resistant to destruction while preserving the contents of the interior and maintaining sterility within.

Exterior Surface: In the arrangement shown, as one example, and with reference to the figures, the main body 20 of system 10 is generally flat and rectangular in shape. The exterior surface 22 of the main body 20 is generally smooth with ridges 29 located on the cover 28. The cover 28 forms the front portion of the main body 20 in a way that the cover 28 conceals the hollow interior 26 of the main body 20. Similarly, in the arrangement shown, the back 30 of the main body 20 may include ridges 33. Ridges of the front and the back may be formed of any suitable size shape and design and are configured for ease of carrying and use to reduce on slipping of the exterior surface 22 of the main body 20.

Opposing Sides: Additionally, and in the arrangement shown, as one example, the main body 20 includes opposing sides 32. Opposing sides 32 may be formed of any suitable size, shape, and design and are configured to form the side portions of the carry case 12. In the arrangement shown, as one example, opposing sides 32 are in situated in a parallel spaced relation to one another on opposite sides of the carry case 12. In this way, opposing sides 32 form two of the main 4 sides of the main body 20. In the arrangement shown, as one example, opposing sides 32 also include a plurality of ridges 33. Plurality of ridges 33 are formed of any suitable size, shape, and design and are configured to aid a user in gripping the main body 20.

Grip: In addition to, an in the arrangement shown as one example, the main body 20 includes a grip 34. Grip 34 is formed of any suitable size, shape, and design and is configured to aid a user in gripping the carrying case 12 and the main body 20. In the arrangement shown, as one example, grip 34 is formed of a unitary construction with insignia and a plurality of ridges which aid in gripping and aesthetic appearance of the carry case 12 and main body 20. In the arrangement shown, grip 34 covers the lower portion of the front of the carry case 12. In this way, the grip 34 is also configured to cover the chamber 40 (to be further discussed herein). Furthermore, the grip 34 covers the opposing sides 32 in a way that aids in carrying and using the carry case 12, dispenser 14, and the entire method of use.

In the arrangement shown, as one example, the grip 34 is formed of a polymer. However, any other material for forming a useful grip 34 which aids in holding and using the system 10 are hereby contemplated for use. Other materials may include enhanced polymers, vinyl, polyvinyl chloride (pvc), metals such as aluminum, steel and the like, metal composites, carbon fiber, as well as other combinations of materials that are resistant to destruction while providing a surface suitable for gripping and creating comfort.

Push Release: In the arrangement shown, as one example, the carry case 12 includes a push release 38. Push release 38 is formed of any suitable size, shape, and design and is configured to be a release button which disengage the interlocking engagement of the bottom 60 of the carry case 12 and the main body 20. In the arrangement shown, as one example, the push release 38 is a frictional fit interaction between the opposing sides 32 and the front cover 28. In this way, the push release 38 allows the front cover 28 to slidably disengage being locked in place. Additionally, although a frictional fit is used in the arrangement herein, other forms of engagement are hereby contemplated for use, such as clipping, interlocking mechanisms, hinged connection, and more.

In the arrangement shown, as one example, the push release 38 is generally rectangular in shape and is an elongated square extending almost entirely across the front surface of the cover of the main body 20 of the carry case 12. However, the push release 38 may be formed of any suitable size, shape and design. Therefore, the push release 38 may be a small circular button, may be a type of lever, may be a type of quick release or may be spring loaded, or may be a plurality of ridges with an intuitive design. These and other designs and sizes for a push release 38 are hereby contemplated for use.

In the arrangement shown, as one example, when the push release 38 is pressed and the front cover is caused to slide downwardly away from the lid, the compartment 68 of the bottom 60 is exposed as the entire bottom 60 and front cover slide downward. Additionally the hollow interior 26 of the main body 20 of the carry case 12 is exposed along with the plurality of chambers 40 within.

### Chamber

In the arrangement shown, as one example, the main body 20 of the carry case 12 includes a plurality of chambers 40. Plurality of chambers 40 are formed of any suitable size, shape, and design and are configured to accept, store, and dispense a plurality of hearing protection devices. In the arrangement shown, as one example, a chamber 40 includes a top 42, a bottom 44, a hollow tube 46, a step 48, a compression clip 50, a spring 52, and a spring support 54, among other components and features.

In the arrangement shown, as one example, 4 chambers are configured in the hollow interior 26 of the main body 20 of the carry case 12. These 4 chambers are generally each an elongated chamber extending almost the entire length of the hollow interior 26 of the main body 20 of the carry case 12. In this way, each chamber 40 is accessible by the applicator 90 (to be further discussed herein) from the top of the chamber. Said another way, each chamber is accessible at the portion of the chamber 40 located nearest the door 70 (to be further discussed herein). From the top 42 of the chamber 40, each chamber extends a length to a bottom 44.

Hollow Tube: In the arrangement shown, as one example, each chamber 40, extending a length, has a portion forming the upper part of the length which is a hollow tube 44. Hollow tube 44 is formed of any suitable size, shape, and design and is configured to accept a hearing protection device 18 therein. In this way, the hollow tube 44 is the storage location for a single hearing protection device 18.

In the arrangement shown, as one example, a hearing protection device 18, or more specifically a plug 120 (to be further discussed herein) is placed within hollow tube 44. Subsequently, the plug 120 is compressed by the compression clip 50. Once the compression clip 50 has compressed the plug 120, an applicator 90 can be used to push against the step 48 of the chamber 40. In this way, the hollow tube 44 is pressed away while the compressed plug 120 is forced out of the hollow tube 44 and into the hollow interior 102 of the applicator 90. (This process is further discussed herein; see method of use).

Step: In the arrangement shown, as one example, each chamber 40 also includes a step 48. Step 48 is formed of any suitable size, shape, and design and is configured to aid in transferring the plug 120 from the hollow tube 4 to the hollow interior 102 of the applicator 90. Step 48 is a circular depression around the perimeter of the top opening of the hollow tube 44. In this way, step 48 is designed to fit the circumference of the end of applicator 90. In this way, when the applicator 90 is pressed against the top of the chamber 40, the applicator fits within the step 48 of the chamber 40.

Said another way, as the applicator 90 engages the step 48, the applicator 90 can be used to press the hollow tube 44 portion of the chamber 40 downward engaging the spring 52 while the spring support 54 (or “slide”) forces the plug 120 into the hollow interior 102 of the applicator 90. In this way, the slide 54, which is sized and shaped to fit within the hollow tube 44 moves into the hollow tube 44 when the applicator 90 forces the hollow tube 44 downward and engages the spring 52. Said another way, the slide 54, which simultaneously supports the spring 52 by being sized to fit within the spring 52, is forced into the interior of the hollow tube 44. Said yet another way, when the applicator 90 presses downward on the hollow tube 44, the hollow tube 44 engages the spring 52 below and beings to slide downward. The spring support 54, which is stabilized in place, does not slide downward. Instead the spring support 54 is forced into the hollow interior of the hollow tube 44 which forces the compressed plug 120 to be displaced out of the hollow tube 44. In this way the plug 120 doesn't move downward like the hollow tube 44. In this way, the plug 120 is enters the hollow interior of the applicator 90.

In the arrangement shown, as one example, four chambers 40 fill the hollow interior 26 of the main body 20 of carry case 12. However, any other number of chambers 40 is hereby contemplated for use. Other numbers of chambers include a single chamber 40, two chambers 40, three chambers 40, five chambers 40, six chambers 40, seven chambers 40, eight chambers 40, nine chambers 40, or more chambers 40.

Bottom: In the arrangement shown, the carry case 12 includes a bottom 60. Bottom 60 is formed of any suitable size, shape, and design and is configured to form the bottom portion of the carry case as well as store extra plugs 120. Bottom 60 includes an exterior surface 62, an interior surface 64, and a hollow interior 66 which makes up a compartment 68 for storing plugs 120 therein. In the arrangement shown, as one example, the bottom 60 is a separate component of the carry case 12, or system 10, which is slidably connected to the main body 20. In this way, the bottom 60 slides downward to expose a small compartment 68 which is generally located behind the grip 48 when the bottom 60 is engaged, or said another way, is closed.

In the arrangement shown, as one example, the bottom also forms the cover 28. The bottom, in this way, also serves as the force compressing the plugs 120 when the plugs are in the hollow tube 44. In action when the bottom is open, the compression clips 50 are disengaged or opened outward, out of the hollow interior 26 of the main body 20. Said another way, and with reference to the figures, the compression clips 50 are disengaged when the bottom 60 is open. When the bottom 60 is forced closed, the compression clips are returned to the closed position such that the compression clip is able to compress a plug located within the hollow tube 44.

Door: In the arrangement shown, as one example, carry case 12 includes a door 70. Door 70 is formed of any suitable size, shape, and design and is configured to store the application 90 within its hollow interior 76. Additionally, door 70 is configured to provide access to the dispenser portion or hollow interior of the carry case. In this way, the applicator 90 and the interior, which is used to dispense plugs 120 are accessible by opening one door 70. In the arrangement shown, as one example, door 70 includes an exterior surface 72, an interior surface 74, a hollow interior 76, a front 78, a back 80, opposing sides 82, an open release 84, connectors 86 (or “fasteners”), and an applicator support feature 88, among other components and features.

In the arrangement shown, as one example, the door 70 is formed of a polymer. However, any other material for forming a useful carry case and dispenser are hereby contemplated for use. Other materials may include enhanced polymers, vinyl, polyvinyl chloride (pvc), metals such as aluminum, steel and the like, metal composites, carbon fiber, as well as other combinations of materials that are resistant to destruction while preserving the contents of the interior and maintaining sterility within.

Exterior Surface: In the arrangement shown, as one example, and with reference to the figures, the door 70 of the main body 20 of the carry case 12 and dispenser 14 of system 10 is generally barrel like in shape having a semi-circular shape extending the length of the carry case 12 between opposing ends. The exterior surface 72 of the door 70 is generally smooth. Similarly, the interior surface 74 is generally smooth.

Opposing Sides: Additionally, and in the arrangement shown, as one example, the door 70 includes opposing sides 82. Opposing sides 82 may be formed of any suitable size, shape, and design and are configured to form the side portions of the door 70. In the arrangement shown, as one example, opposing sides 82 are situated in a parallel spaced relation to one another on opposite sides of the door 70, forming a part of the door 70 thereof. In the arrangement shown, as one example, opposing sides 82 also include a plurality of ridge like structures which aid in gripping the door 70 and add stability to the door structure 70.

Open Release: In the arrangement shown, as one example, the door 70 includes an open release 84. Open release 84 is formed of any suitable size, shape, and design and is configured to be a release button which disengage the interlocking engagement of the door 70 from the carry case 12 and the main body 20. In the arrangement shown, as one example, the open release 84 is a frictional fit interaction between the opposing sides 82 and the main body 20. In this way, the open release 84 allows the door 70 to frictionally disengage being locked in place and swing open about a soft connection or hinged connection. Additionally, although a frictional fit is used in the arrangement herein, other forms of engagement are hereby contemplated for use, such as clipping, interlocking mechanisms, hinged connection, and more. Furthermore, as a soft connection, or section of weakened material is used so that the door can rotate about an axis and open, other connections are hereby considered for use. While connectors 86 are used in the example shown, other connections may be a hinged connection, a spring connection, a joint connection, or other connections which allow for a door to open.

In the arrangement shown, as one example, the open release 84 is generally rectangular in shape and is an elongated square extending almost entirely across the front surface of the cover of the front of the door 70 of the carry case 12. However, the open release 84 may be formed of any suitable size, shape and design. Therefore, the open release 84 may be a small circular button, may be a type of lever, may be a type of quick release or may be spring loaded, or may be a plurality of ridges with an intuitive design. These and other designs and sizes for a open release 84 are hereby contemplated for use.

In the arrangement shown, as one example, when the open release 84 is pressed and the door 70 (or lid) is caused to swing upward away from the main body 20, the dispenser is exposed. Additionally the hollow tube 44 of the chambers 40 is exposed along with the plurality of steps 48 within.

Applicator Support: In the arrangement shown, as one example, the door 70 includes an applicator support 88. Applicator support 88 is formed of any suitable size, shape, and design and is configured to securely attach (or hold) an applicator 90 within the hollow interior 76 of the door 70. In the arrangement shown, as one example, applicator support 88 is formed of a plurality of rigid members extending perpendicular to the interior surface of the door 70. Additionally, the plurality of rigid members include a recess sized and shaped to receive an applicator 90, therein.

In the arrangement shown, as one example, applicator supports 88 are formed of a plurality of rigid members. In this way, the applicator supports 88 shown in the example work by frictional fit by engaging the exterior surface of the applicator 90. However, other applicator support 88 and/or applicator securement features are hereby contemplated for use. Additionally, the hollow interior without any applicator supports 88 may suffice as a featured storage area of an applicator 90. Other applicator support 88 features may include, but are not limited to a hook and ladder feature, a magnetic feature, a moveable hinge feature, or other engagement mechanism which holds the applicator 90 in place within the hollow interior of the door 70 of system 10.

Applicator: In the arrangement shown, as one example, system 10 includes an applicator 90. Applicator 90 is formed of any suitable size, shape, and design and is configured to accept a plug 120 therein, hold a plug 120 therein, and dispense a plug 120. More specifically, and without limitation, applicator 90 as disclosed herein is designed to inject or apply a plug 120 within the ear canal of a user such that the plug 120 is used for noise cancellation. In this way, a plug 120 can be used as a hearing protection device.

In the arrangement shown, as one example, applicator 90 includes a top 92, a bottom 94, a sidewall 96 (or “rigid sidewall”), an exterior surface 98, an interior surface 100, a hollow interior 102, a plurality of grip features 104, a plunger 106, and at least one interlocking step 108, among other features or components.

Plug: In the arrangement shown, as one example, system 10 includes a plug 120. Plug 120 is formed of any suitable size, shape, and design and is configured to create an airtight seal in the ear canal of a user. In this way, the plug forms a seal capable of cancelling some, or most, sound waves caused by an environment. In other words, plug stops sound waves from entering the ear canal, such that noise is reduced or eliminated. Plug 120 may also be known as a hearing protection device.

In the arrangement shown, as one example, plug 120 is a foam, tapered bullet shaped device capable of expanding and contracting when forced to contract. Plug 120, once contracted or forced to take up less volume area, then expands when forces are removed. In the arrangement shown, as one example, plug 120 is generally bullet shaped and extends from a top 122, or interior end/insertion end, to a bottom 124, or exterior/outside end and includes a tapered sidewall 126.

### Alternative Embodiment

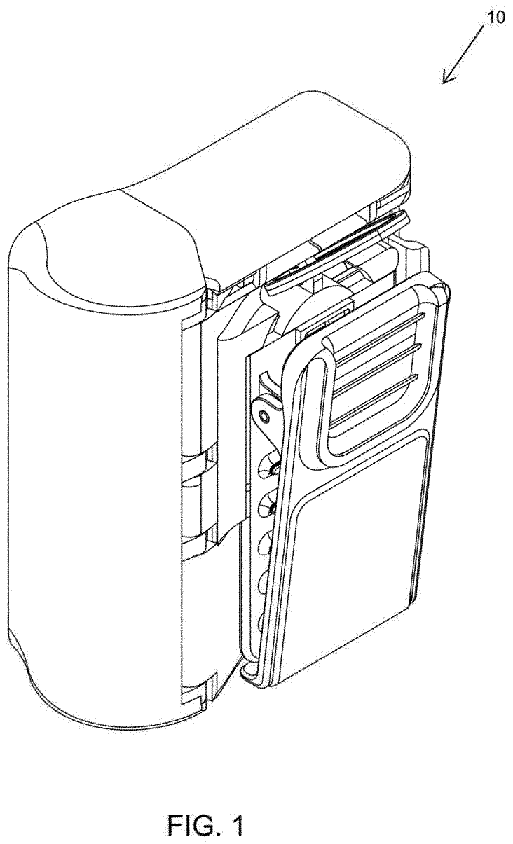

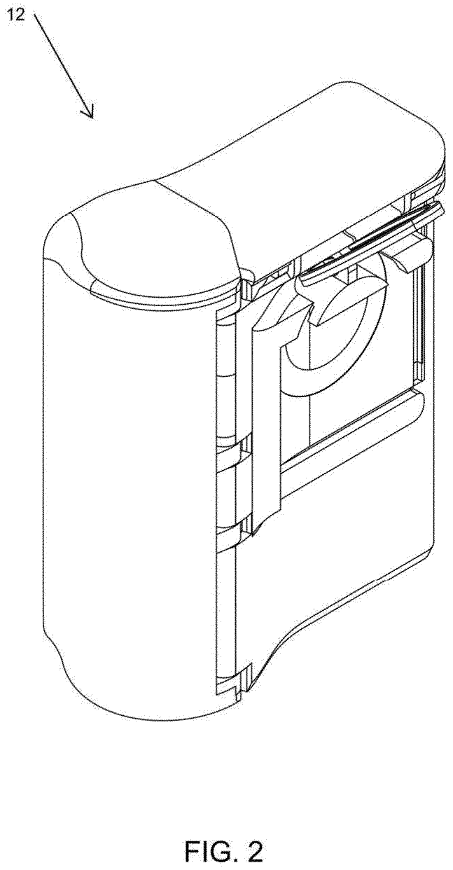

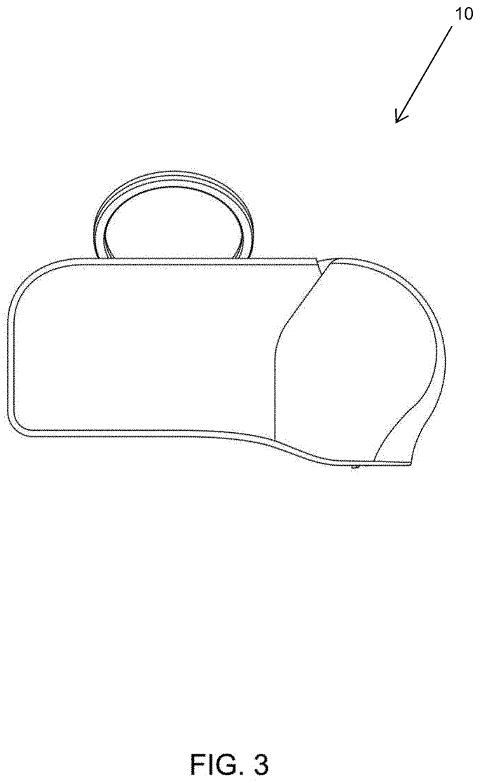

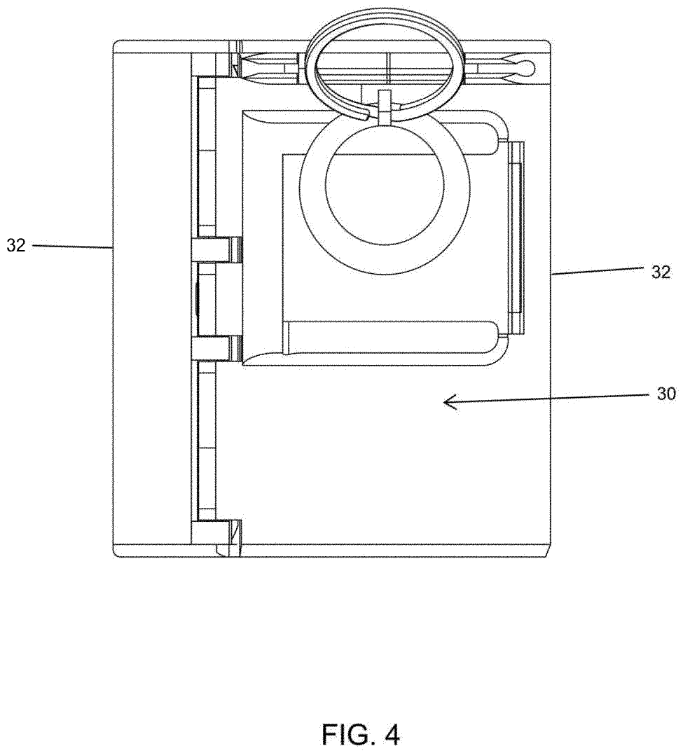

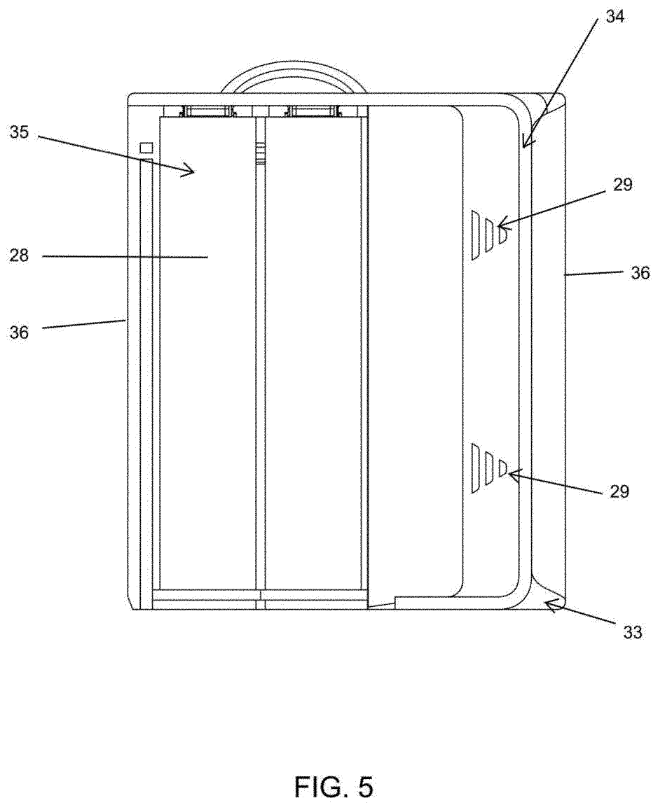

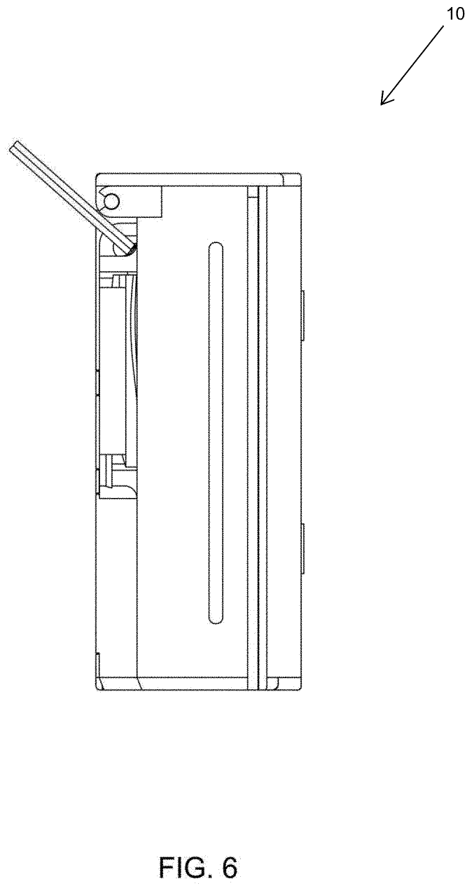

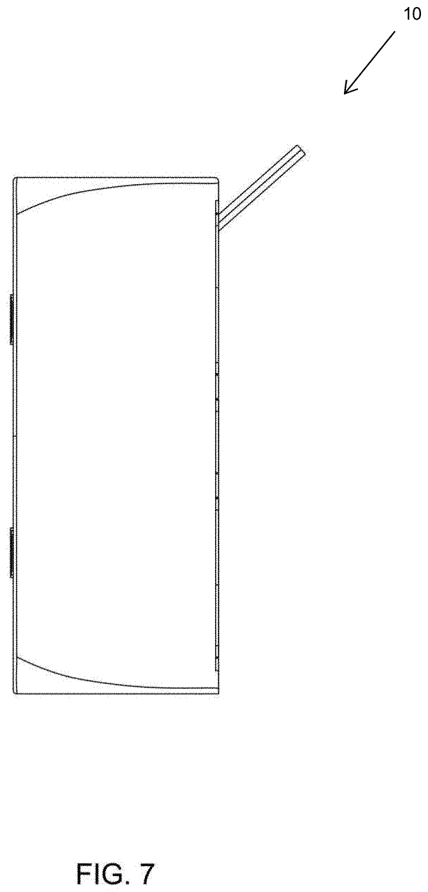

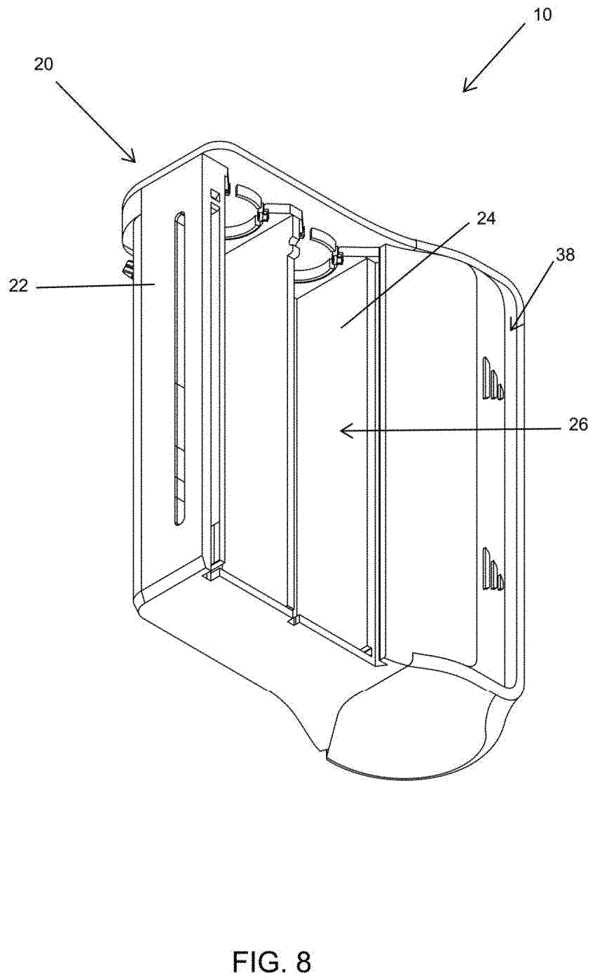

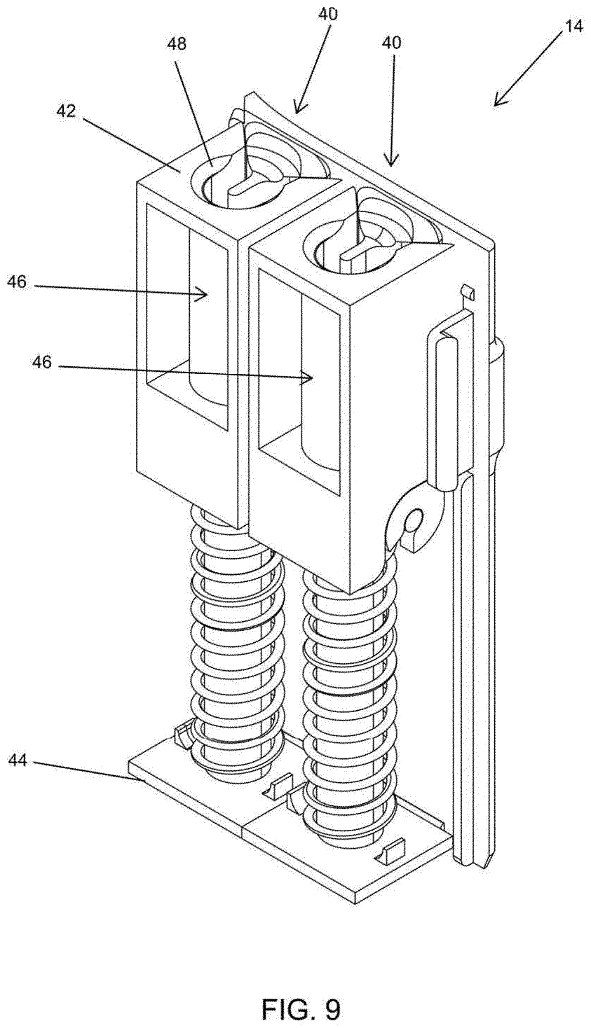

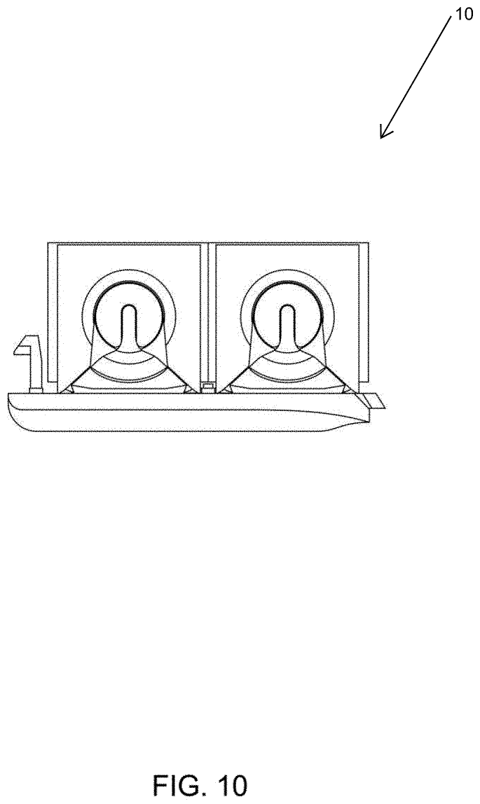

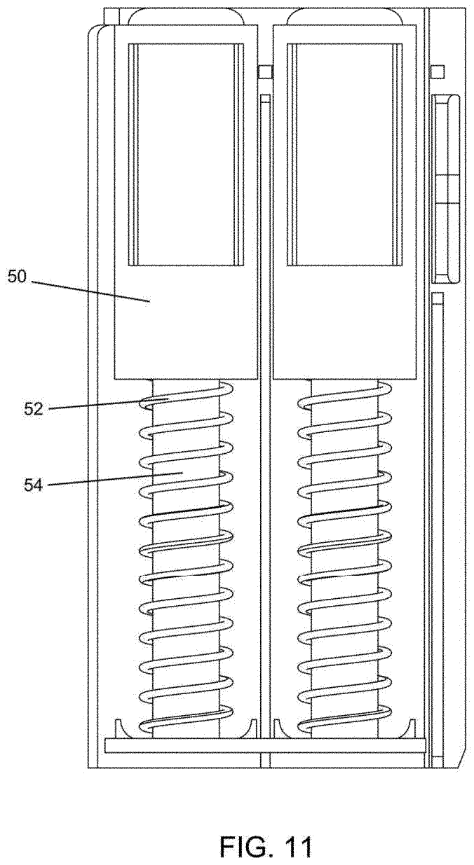

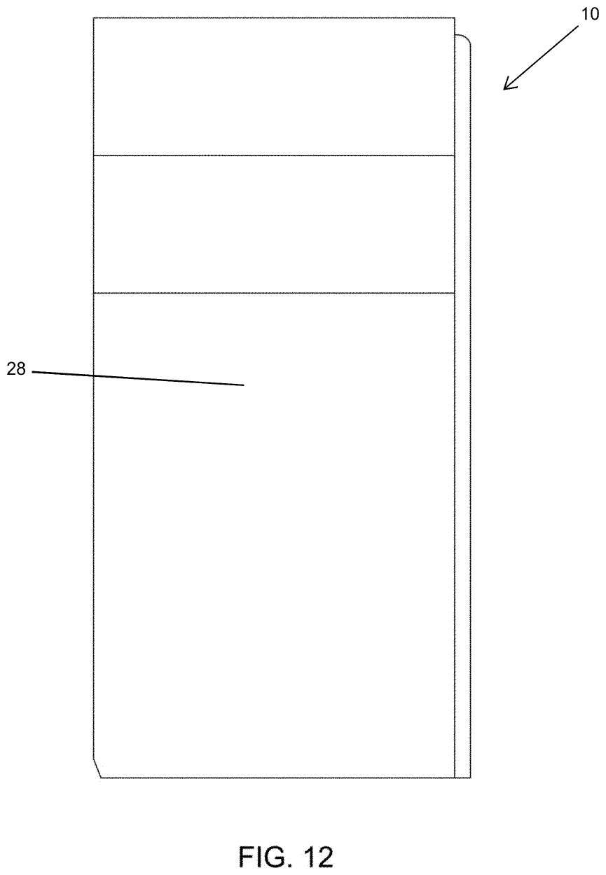

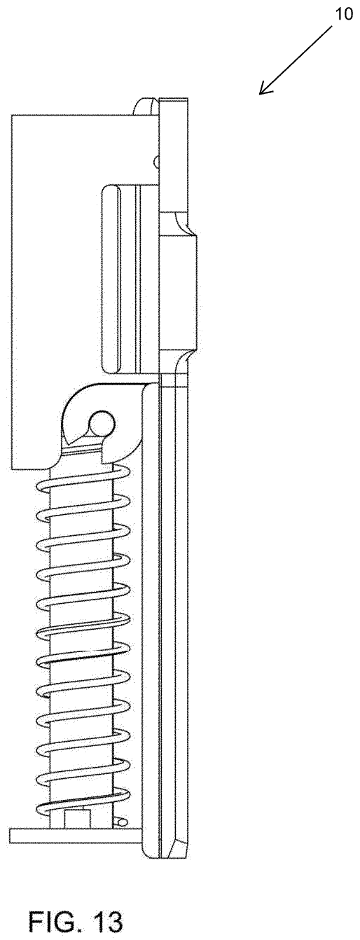

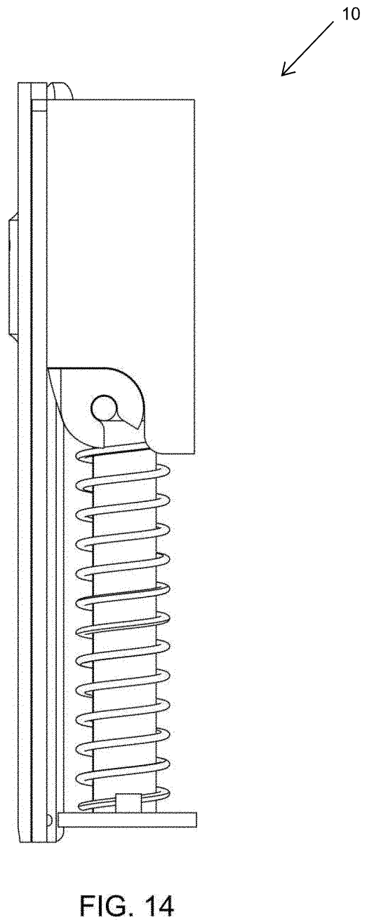

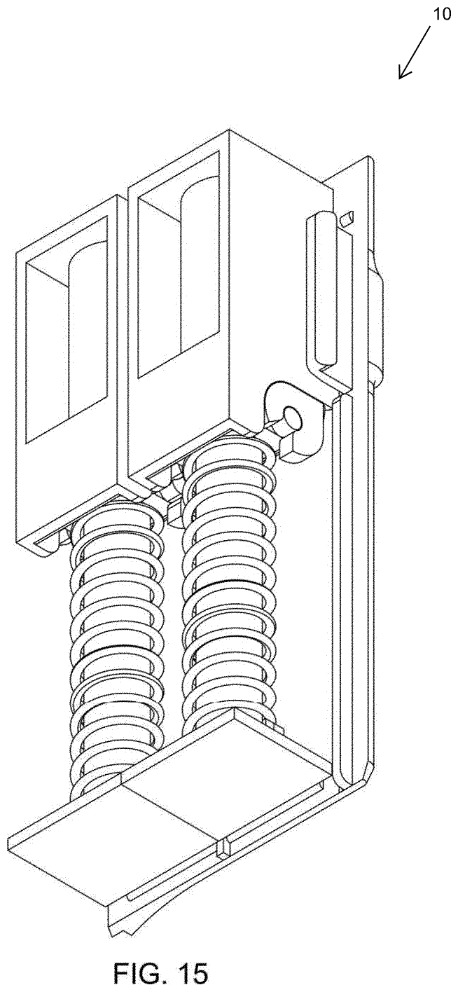

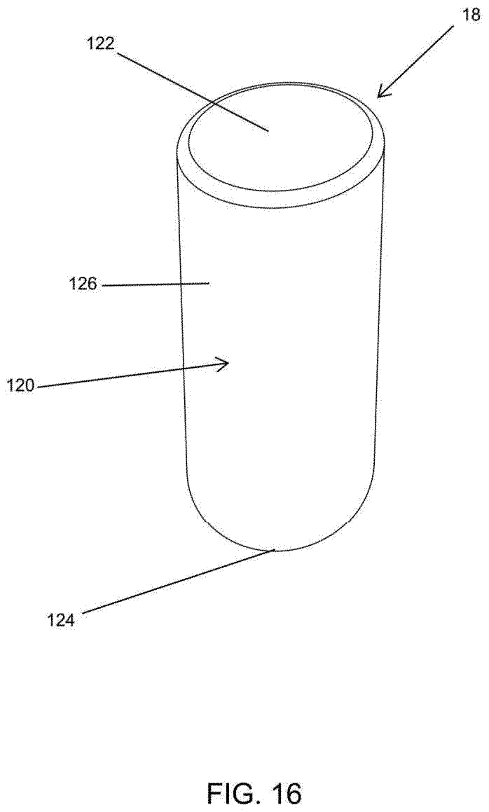

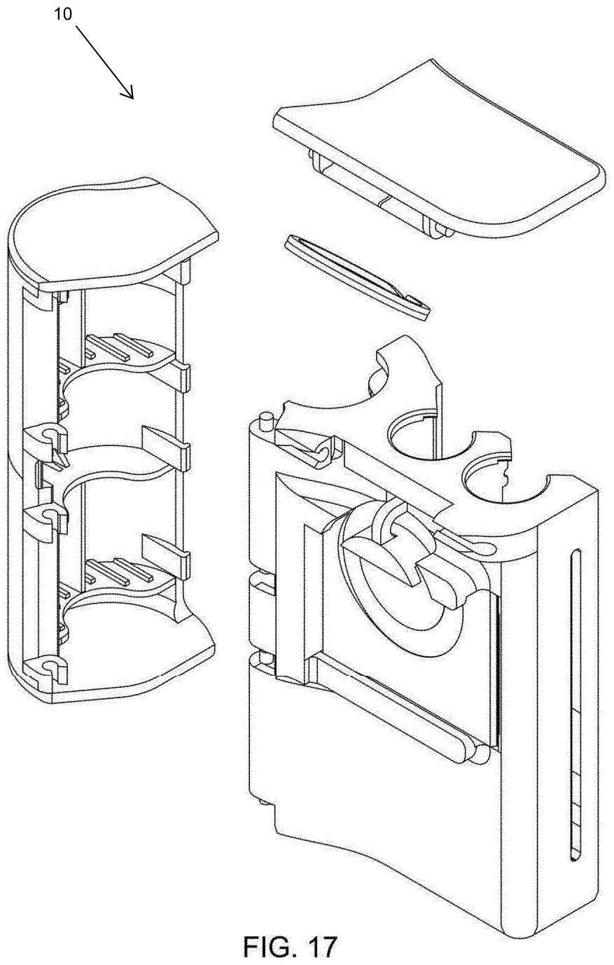

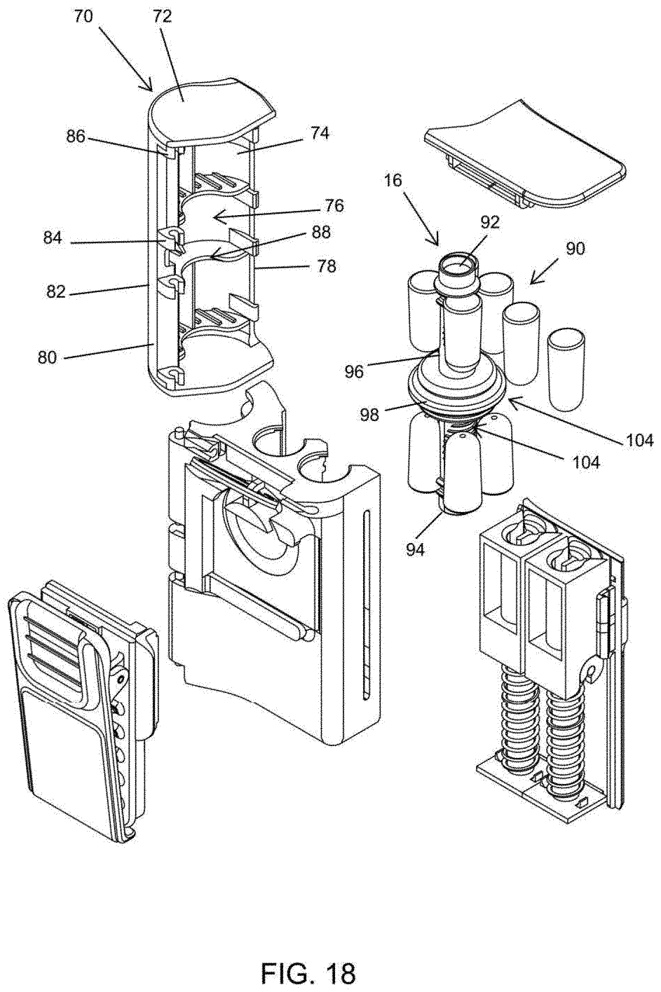

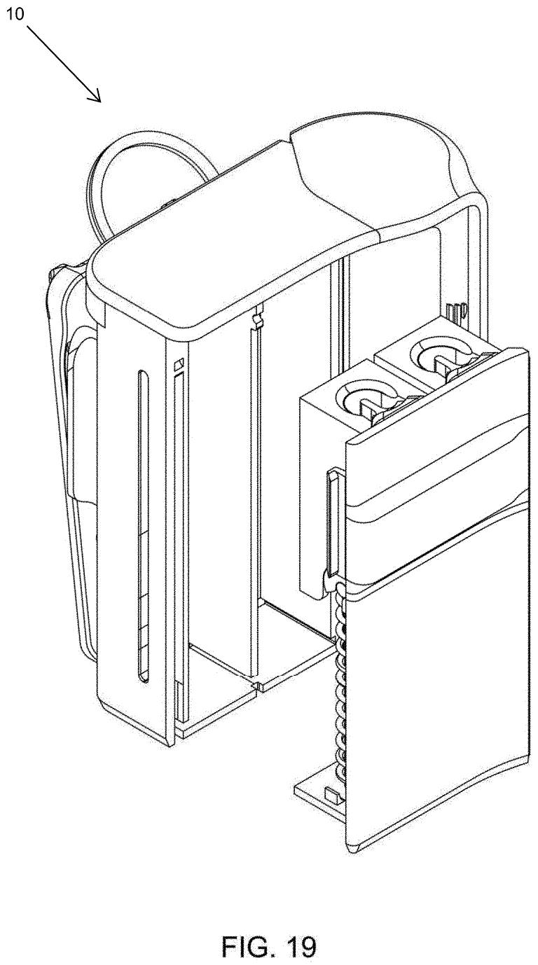

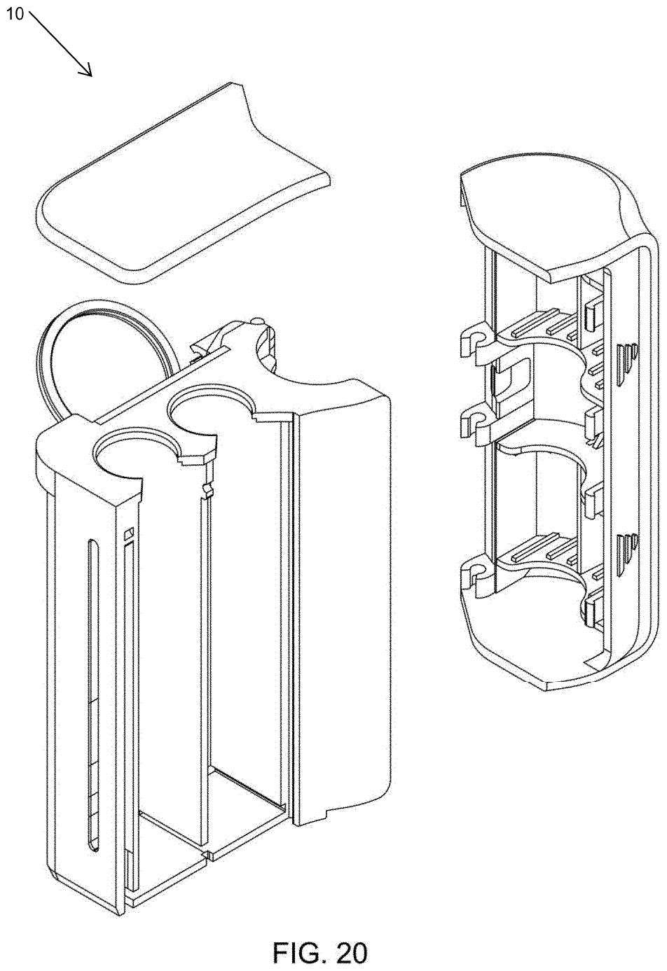

In one arrangement, and as is shown in FIGS. 1-20, the chamber for storage of the hearing protection device and applicator is located along the side of the main body. In this arrangement, and in this way a swing door serves as access to the applicator and the plurality of hearing protection devices within.

### IN OPERATION/METHODS OF USE

### System Embodiments

As one example, and in the arrangement shown, a complete system 10 includes a carry case with storage, a dispenser, an applicator, and a hearing protection device (or “plug”).

Loading the Carry Case: In one example, the first step in using the carry case is to load the compartment of the carry case with a plurality of plugs. The first step in this process is to press the push release of the carry case. The push release slides the cover and bottom down from the carry case causing a separation and revealing the compartment for storage in the bottom. Once the bottom is open, a plurality of plugs can be placed within the compartment and the bottom can be returned to a closed position.

Loading the Chamber: The second step in this example of a method of use is to load the hollow tube of the chamber. One or a plurality of hollow tubes may be loaded. Each hollow tube, in the arrangement shown as one example, is to be loaded within a single plug. In order to load a plug in the hollow tube of a chamber, the user needs to first open the bottom of the carry case, if the bottom is not already open. This is done by using the push release and sliding the front cover downward, revealing the hollow interior of the main body of the carry case and the plurality of chambers within.

Once the interior is opened, the compression clip for the chamber needs to be opened as well. The compression clip swings open outwardly revealing the hollow tube of the chamber. After the compression clip has been disengaged from the hollow tube, the plug can be placed within the hollow tube. This is done by placing a single plug within a single hollow tube of a single chamber. A plurality of plugs may be placed within a plurality of hollow tubes within chambers. In an alternative embodiment, it may be possible to place more than one plug in a single hollow tube such that a single applicator can be loaded with multiple plugs. As one example, two plugs might be placed in a single hollow tube such that a plug can be loaded for each of two ears of a user.

After a plug is placed within the hollow tube of the chamber, the compression clip needs to be closed. This is accomplished by manually closing the compression clip, or alternatively, by sliding the bottom shut. The cover of the main body of the carry case will force the compression clip closed if the compression clip is not already closed. When the compression clip closes, this will press forces against the plug and cause the plug to compress, taking up less volume area. With the hollow tube loaded, and the compressed plug or plugs within, the subsequent step is loading the applicator.

In this arrangement, and in the example shown, the plug should be loaded within the hollow tube of the chamber with the tapered end of the plug facing downward. Subsequently this results in correct orientation when the plug is inserted into the ear of a user. This results in correct use of the hearing protection device.

Loading the Applicator: With the plug or plugs loaded within the carry case and ready to be dispensed, the next step is to access the dispenser. The first step in accessing the dispenser is to open the lid or door of the system. The door is open by using the open release. The open release causes the door to open, revealing both the applicator and dispenser and/or top portion of the chambers within the carry case.

With the door open, the applicator first needs to be removed from the applicator supports within the door. Once the applicator is free of the door, the applicator should be used to engage with the dispenser. By placing the interlocking step in line with the step of the top portion of the hollow tube, a user engages the applicator with the dispenser.

Once the applicator is engaged with the dispenser, the applicator should be used to press the hollow tube downward. The hollow tube is pressed downward, and this downward force loads the spring below. Simultaneously, the spring support displaces the plug from within the hollow tube to the interior of the applicator. In this way, the plug remains in place while the hollow tube is removed from the surrounding of the plug. The applicator replaces the position of the hollow tube and thus the applicator becomes loaded with a plug.

Ear Canal Insertion: With the plug in place within the applicator, the applicator can then be used to place a plug within the ear canal of a user. A user next places, the applicator interlocking step against the exterior of the ear canal and injects and/or inserts the plug within an ear canal. Holding the applicator in place, the user grips the applicator and presses the plunger which forcibly ejects the plug into the ear canal. Once the plug is injected into the ear canal and completely free of the applicator, the plug will expand to create an airtight seal in the ear canal. In this way, the hearing protection device will prevent sound and/or sound waves from entering the ear canal.

In the arrangement shown, as one example, this process can be repeated for each ear of the user. In an alternative embodiment, after the first plug is inserted into the first ear, a second plug can be inserted into a second ear by loading the double applicator with two plugs.

It will be appreciated by those skilled in the art that other various modifications could be made to the device without parting from the spirit and scope of this disclosure. All such modifications and changes fall within the scope of the claims and are intended to be covered thereby.

What is claimed:

1. A hearing protection device system, comprising:
a carry case;
the carry case having a main body extending a length from a top to a bottom between opposing sides;
the carry case having a hollow interior;
a dispenser;
the dispenser extending a length from a top to a bottom;
the dispenser having a plurality of hollow tubes;
the dispenser having a compression clip;
each of the hollow tubes having a spring support;
each of the hollow tubes having a spring;
wherein the dispenser is configured within the hollow interior of the carry case;
a plug;
the plug extending a length from a first end to a second end;
wherein the plug is placed within the hollow tube of the dispenser;
wherein the compression clip causes the plug to be formed into a particular position;
wherein the hollow tube of the dispenser is operably connected to the spring support and is configured to slide up and down the exterior surface of the spring support;
wherein the spring is configured outside of the spring support;
wherein sliding the compression clip down the spring support causes the spring to become compressed;
wherein sliding the compression clip down causes the spring support to push the plug out of the top of the compression clip.

2. The system of claim 1, further comprising:
a compartment;
wherein the chamber provides storage for at least one plug.

3. The system of claim 1, further comprising:
a compartment;
the chamber extending a length from a first end to a second end;
wherein the compartment is located along one of the opposing sides of the main body;
wherein the chamber provides storage for at least one plug.

4. The system of claim 1, further comprising:
a compartment;
the chamber extending a length from a first end to a second end;
wherein the compartment is located along one of the bottom of the main body;
wherein the chamber provides storage for at least one plug.

5. The system of claim 1, further comprising:
a compartment;
wherein the chamber provides storage for at least one plug.
wherein the compartment is operably connected to the main body;
where the compartment can move from an open position to a closed position.

6. The system of claim 1, further comprising:
a compartment;
wherein the chamber provides storage for at least one plug;
wherein the compartment has a plurality of ridges;
wherein the compartment has a frictional fit closing and opening feature.

7. The system of claim 1, further comprising:
a front cover.

8. The system of claim 1, further comprising:
a front cover;
wherein the front cover is operably connected to the main body;
wherein the front cover is configured to move the compression clip into an open position when the front cover is moved to an open position.

9. The system of claim 1, further comprising:
a front cover;
wherein the front cover is operably connected to the main body;
wherein the front cover is configured to move the compression clip into a closed position when the front cover is moved to a closed position.

10. The system of claim 1, further comprising:
a front cover;
wherein the front cover is operably connected to the main body;
wherein the front cover is configured to move the compression clip into an open position when the front cover is moved to an open position;
wherein the front cover is configured to move the compression clip into a closed position when the front cover is moved to a closed position.

11. The system of claim 1, further comprising:
an attachment feature;
wherein the attachment feature is operably connected to the main body of the carry case.

12. The system of claim 1, further comprising:
a lid;
wherein the lid is operably connected to the main body of the carry case.

13. A hearing protection device, comprising:
a carry case;
the carry case having a main body extending a length from a top to a bottom between opposing sides;
the carry case having a hollow interior;
a dispenser;
the dispenser extending a length from a top to a bottom;
the dispenser having a plurality of hollow tubes;
the dispenser having a compression clip;
each of the hollow tubes having a spring support;
each of the hollow tubes having a spring;
wherein the dispenser is configured within the hollow interior of the carry case;
a plug;
the plug extending a length from a first end to a second end;
wherein the plug is placed within the hollow tube of the dispenser;
wherein the compression clip causes the plug to be formed into a particular position;
wherein the hollow tube of the dispenser is operably connected to the spring support and is configured to slide up and down the exterior surface of the spring support;
wherein the spring is configured outside of the spring support;
wherein sliding the compression clip down the spring support causes the spring to become compressed;
wherein sliding the compression clip down causes the spring support to push the plug out of the top of the compression clip;
an applicator;
the applicator extending a length from a first end to a second end;
wherein at least part of the applicator is formed of a hollow interior;
wherein the applicator accepts the plug within the hollow interior of the applicator.

14. The system of claim 13, further comprising:
the applicator having a step feature;
the compression clip having a step feature located at the top of the hollow interior of the compression clip;
wherein the applicator step is configured to fit within the step of the compression clip.

15. The system of claim 13, further comprising:
wherein the applicator has a plunger feature;
wherein the plunger feature is configured to press a plug out of the hollow interior of the applicator.

16. The system of claim 13, further comprising:
a compartment;
wherein the chamber provides storage for at least one plug.

17. The system of claim 13, further comprising:
a front cover;
wherein the front cover is operably connected to the main body;
wherein the front cover is configured to move the compression clip into an open position when the front cover is moved to an open position;
wherein the front cover is configured to move the compression clip into a closed position when the front cover is moved to a closed position.

18. The system of claim 13, further comprising:
a lid;
wherein the lid is operably connected to the main body of the carry case.

19. A method of blocking sound, the steps comprising:
providing a carry case, a dispenser, and a plug; wherein the carry case houses a dispenser within the hollow interior of the carry case; the dispenser having a spring support and a spring and a compression clip; the carry case having a front cover;
sliding the front cover downwards; wherein opening access to the hollow interior of the carry case;
opening the compression clip;
inserting the plug into a hollow interior of the compression clip;
closing the compression clip; wherein the compression clip closing causes the plug to become formed;
dispensing the formed plug;
inserting the formed plug into the ear canal of a user.

20. The method of claim 19, further comprising:
providing an applicator;
dispensing the plug from the dispenser into a hollow interior of the applicator;
pressing the plug into an ear canal of a user by pressing a plunger of the applicator.
